(19) United States
(12) Patent Application Publication
(10) Pub. No.: US 2006/0166363 A1
(43) Pub. Date: Jul. 27, 2006

(54) Modified baculovirus expression system for production of pseudotyped rAAV vector

(76) Inventors: Sergei Zolotukhin, Gainesville, FL (US); Nicholas Muzyczka, Gainesville, FL (US); Erik Kohlbrenner, Gainesville, FL (US)

Correspondence Address: EDWARDS & ANGELL, LLP, P.O. BOX 55874, BOSTON, MA, 02205, US

(21) Appl. No.: 11/043,658

(22) Filed: Jan. 26, 2005

Related U.S. Application Data
(60) Provisional application No. 60/539,660, filed on Jan. 27, 2004.
(60) Provisional application No. 60/612,066, filed on Sep. 22, 2004.

(51) Int. Cl. C12N 15/861; C12N 15/86; C12N 5/06

(52) U.S. Cl. 435/456; 435/348

(57) ABSTRACT

The invention provides modifications to a baculovirus-based recombinant adeno associated virus (AAV) system including enhancement of the helper virus stability and construction of novel baculovirus vectors for rAAV pseudotyping. The modified system extends the flexibility of rAAV vector production and promotes the utility of AAV as, a clinically applicable gene therapy vector.

### REFERENCE TO RELATED APPLICATIONS

This application claims benefit to provisional patent application Ser. No. 60/539,660 filed Jan. 27, 2004 and provisional patent application Ser. No. 60/612,066 filed Sep. 22, 2004.

The United States Government has certain rights in the present invention pursuant to grants DK62302, HL59412, and DK58327 from the National Institutes of Health.

### BACKGROUND OF THE INVENTION

Scalable production of rAAV vectors remains a major obstacle to the clinical application of this prototypical gene therapy vector. A recently developed baculovirus-based production protocol found limited application due to the system design (Urabe, et al, Mol Ther 9:S160, (2004). Unfortunately, stability problems exist with this system in for use in scalable production.

Viral vectors have become vectors of choice for gene delivery. Gene transfer is employed for delivery of therapeutic protein encoding nucleic acids to target cells. The DNA may encode one or more genes desired to be express in a target cell and the sequences controlling expression of the gene(s). Therapeutic applications require transportation via vectors that internalize to a cell after binding to the cell membrane. After transportation into the cell nucleus, the genome is integrated into the cell nucleus or, depending on the vector, exists in the nucleus as an eipsome.

Commonly used gene transfer vectors include liposomes, molecular conjugates, retroviruses, adenoviruses (Ad) and adeno-associated viruses (AAV), of which Ad and AAV have been most extensively studied. Less extensively studied are herpes, cytomegalovirus, poxvirus, vaccinia, lentiviral and baculovirus.

While adeno viruses have been extensively studies, the more promising gene vectors are adeno-associated viruses (AAV). The AAVs transduce non-dividing cells and have demonstrated lasting gene expression in a wide spectrum of tissue types. Perhaps the most important drawback to their use is that they are difficult to produce and have a relatively small delivery capacity. Approximately 5 kb is about the limit that can be placed in an expression cassette.

Recombinant adeno-associated virus (rAAV) vector has emerged recently as one the most versatile gene therapy delivery vehicles. The mainstream utility of rAAV derives in part from the natural plasticity of its structural and regulatory viral components. AAV genomes are widely disseminated in human and nonhuman primate species, with rapid molecular evolution resulting in the formation of quasi-species and novel, serologically distinct serotypes (Gao, et al., Proc Natl Acad Sci USA 100:6081-6 (2003)); (Gao, et al., 2004, J Virol 78:6381-8).

Taking advantage of the structural relationships among the diverse serotypes, investigators have been able to exploit their modular nature by combining specific vector components derived from each serotype. Using the processes dubbed “pseudotyping” (Hildinger, et al., J Virol 75:6199-203(2001)), or “cross-packaging” (Rabinowitz, et al., J Virol 76:791-801 (2002)), chimeric vectors can be constructed that contain AAV2-derived terminal repeats harboring transgene packaged into capsids of other AAV serotypes. This approach greatly facilitates vector production and therapeutic screening by allowing the same transgene cassette to be packaged for direct comparison of transduction efficiencies of the targeted tissues based specifically on the composition of the viral particle per se.

The logical extension of this approach was the generation of chimeric rAAVs using “trans capsidation” or “cross-dressing” technique whereby the virion consisted of a random mosaic of capsid proteins derived from two different AAV serotypes combined at different ratios (Hauck, et al., Mol Ther 7:419-25 (2003); Rabinowitz, et al., J Virol 78:4421-32 (2004)). Such mosaic vectors can exhibit dual receptor binding characteristics of the parental viruses, and providing optimal stoichiometry of components, may even display a synergistic effect in transduction.

The agility of AAV vector production has been further improved by (Urabe, et al., Hum Gene Ther 13:1935-43(2002)), who demonstrated the feasibility of producing these vectors in insect cells using a recombinant baculovirus system. While promising for the production of AAV2, this method has not been shown suitable for the production of pseudotyped rAAV vectors in a large-scale format.

Kotin, et al (patent application 20040197895, 2004) have described a method of producing high-titer rAAV vectors in insect cells. Baculovirus vectors that include nucleic acids that encode Rep78/68 and Rep52/40 were constructed in a palindromic head-to-tail arrangement and used in various combinations with an ITR AAV transgene encoding sequence and capsid genes to show feasibility of rAAV production in the insect cells. While high titer rAAV was initially produced, there was no evidence that the method would be adaptable to large-scale production of rAAV.

Adeno associated viruses (AAV) are human parvoviruses that are dependent on a helper virus, usually adenovirus (AV), to proliferate. AAV is non-pathogenic capable of infecting both dividing and non-dividing cells. In the absence of a helper virus, it integrates into a single site of the host genome (19q-13-qter). The wild type AAV genome is a single-stranded DNA molecule containing only two genes; rep, coding for proteins that control replication, integration into the host genome, and structural gene expression; and cap, coding for the capsid structural proteins.

Adeno-associated virus (AAV) vectors have become increasingly popular as vehicles for transfection of mammalian cells, particularly in delivering therapeutic molecules for treatment of diseases and genetically induced disabilities. When used as a vector, the rep and cap genes are replaced by a transgene and its associated regulatory sequences. One disadvantage of AAV vectors is that the insert is limited to about 5 kb, which is the length of the wild type genome.

Nevertheless, a large number of genes have been inserted into the AAV vector, including genes expressing products that have in vivo therapeutic effects; e.g., human erythropoietin, apolipoprotein and Factor IX.

Scalable production of rAAV vectors remains a major obstacle to the clinical application of AAV gene therapy vectors, which are currently considered to be the preferred viral-based delivery vectors. Production of recombinant AAV vectors has become an important area of interest because yields of virions produced by current methods are typically low. Gene therapies may require up to 1×10¹⁵ particles for parenteral administration and high titer stocks are not available from large-scale productions. Supplies are limited and expensive.

In general, production of rAAV vectors utilizes cap and rep genes supplied in trans, in addition to helper virus gene products, E1a, E1b, E2a, E4 and VA RNA, which may be provided from an adenovirus genome. A typical production method is to co-transfect two plasmids into a competent cell line, such as 293 or COS cells. One plasmid contains a recombinant AAV vector encoding a selected transgene between two ITRs and the other a vector encoding rep and cap functions. Other production methods have employed multiple vectors or plasmids, with the rep and cap genes on different vectors. Not all rep genes need be included on the vector in order to obtain efficient replication; at least a “large” (preferably 78 kD) and “small” (preferably 52 kD) Rep protein gene appear to be required.

Virion yields are typically low, on the order of 10³-10⁴ particles/cell. This may be due in some cases to an inhibitory effect by the rep gene product or perhaps to an effect on stoichiometry because rep is supplied in trans without a terminal repeat on the template. Another problem is recombination, resulting in up to 5-10% of wild type AAV in a producer cell.

Low particle yield is a disadvantage in the use of the currently used systems to produce large quantities of infectious rAAV particles. A large number of culture flasks, on the order of hundreds, are required to obtain sufficient quantities of rAAV to use in animal studies and research. High titer and high production methods remain elusive.

Although 293 cells have typically been used to produce rAAV, insect cells have recently received attention. Efficient production has been shown in Sf9 or Sf21 cell lines derived from Spodoptera frugiperda. Urabe, et al., Mol Ther 9:S160, (2004) developed a baculovirus-based production protocol and found limited applications.

Other cell lines can be derived from Drosophila and mosquito species, but so far have not been developed to the point where they have indicated value for large-scale production of rAAV.

### DEFICIENCIES IN THE ART

Unfortunately, helper function provided from vectors containing Rep encoding genes is lost after only a few passages in competent host cells, significantly limiting potential to isolate large quantities of infectious particles. An increase in the number of passages producing high yields of rAAV virions would be of-significant value in developing large-scale production systems that are capable of providing adequate stocks, of rAAVs for gene therapy applications. An improvement in efficient rAAV production would also provide quantities of pseudotyped rAAV, allowing development of gene therapy protocols that, are even more specifically targeted than serotypes currently being tested.

### BRIEF SUMMARY OF THE INVENTION

The present invention addresses some of the problems that have prevented development of a viable large-scale production protocol for rAAV. In particular, methods to alleviate instability problems have been developed by modifying the Rep-encoding component. The work described herein shows that separate vectors for introduction of the AAV Rep protein in rAAV production in insect cells are surprisingly effective in significantly decreasing loss of Rep protein. Loss of this protein in multiple passaging has been a major factor in attempts to develop efficient scale-up procedures. The disclosed Rep expression vectors contribute to efficient, high production of vAAV during multiple passaging in a competent host cell. The use of two separate Rep encoding vectors, respectively encoding a large and a small Rep protein, permits multipassaging without detectable decrease in Rep protein expression. This unexpected result differs significantly from use of a single 52/78 Rep vector that exhibits increased loss of Rep protein expression on multiple passaging. Use of the split Rep-encoding vectors results in little, if any, loss of Rep protein expression after at least five passages.

Modifications to a baculovirus-based rAAV production system have been made, resulting in enhancement of the helper virus stability. The baculovirus vectors are particularly useful for rAAV pseudotyping. Certain modifications include using parvoviral VP1 phospholipase A2 (pvPLA2) motif swapping. The disclosed constructs provide a system that can be readily adapted to large-scale rAAV vector production.

While use of separate Rep vectors provided sustained high titer production of pseudotyped rAAV, the small and large Rep components could also be combined in a single vector, and good results were achieved if the constructs were designed so that the large and small segments were in a tail-to-tail arrangement. This is different from the head-to-tail and

In in vivo experiments, re-designed chimeric rAAV2/8-GFP targeted mainly to the liver, unlike the mammalian cell-derived rAAV8-GFP, which transduced indiscriminately all the tissues tested. This hepatocyte-specific transduction likely resulted from the change in vector tropism, although an overall reduction of VPI PLA2 activity cannot be ruled out.

The results demonstrated that VP1up domains of the AAV viruses are completely modular and can be replaced with homologous domains from other parvoviral capsids, or even with completely un-related phospholipases such as bee venom PLA or PLA of the porcine parvovirus. Such interchangeable PLA modules may be utilized as universal building blocks for novel, highly efficacious vector platforms combining serotype tropism diversity with superior transduction rates. The re-designed baculovirus system disclosed herein improves the capacity for rAAV production by making the AAV platform more amenable to large-scale clinical manufacturing.

A preferred rAAV production protocol employs a four-vector system; i.e., a baculoviral VP vector, a recombinant AAV vector, and separate Rep52 and Rep78 baculovirus vectors.

The total number of viral vectors can also be reduced to three; for example, Bac52 and BacVP or Bac78 and BacVP by placing two open reading frames (ORFs) in tail-to-tail fashion. In making this combination, palindromic sequences similar to a Rep52/Rep78 gene construct reported by Urabe, et al., Gene Ther 13:1935-43 (2002), should be avoided because of lower yields due to loss of Rep on multiple passaging.

An advantage of using three viral vectors is that there is less virus required to propagate and infect the host insect cell, e.g., Sf9 cells, causing less viral load. Additionally, the stoichiometry of the VPs and/or Rep can be changed to optimize rAAV yield.

A surprising advantage of using separate Bac52 and Bac78 vectors is the ability for multiple passaging without a detectable decrease in Rep protein expression. In one example, Sf9 cells were infected at MOI of 5 with four vectors; Bac52, Bac78, BacVP and an rAAV vector. rAAV particle production exceeding 5×10⁴ particles/cell was maintained through at least 5 passages. While similar particle production after a single passage has been reported for production of AAV in insect cells Kotin, et al., (WO 03/042361, published May 22, 2003), the use of Bac52/78Rep leads to almost complete lack of Rep expression after the second passage. Examination of the reported Bac52/78 construct shows a vector constructed with two ORFs coding for large Rep78 and small Rep52 arranged in a tail-to-tail fashion, leading to instability and subsequent deletion within one molecule. The instability appears also to increase recombination events.

The multipassaging advantage over other reported production systems in baculovirus cells is achieved by employing the redesigned vectors herein described, allowing use for large-scale production. Employing the redesigned vectors provides sufficient “active” Rep-expressing baculovirus helper stock to easily infect 10¹⁰ cells in a bioreactor. The new vectors are stable for at least five consecutive passages, which is more than adequate for a bioreactor scale.

Accordingly, while the disclosed vectors have single passage titers similar to those reported with the comparison vector of Urabe, et al. (2002) they exhibit significantly increased stability throughout multiple passaging and provide a practical means to manufacture the quantities of rAAV required for therapeutic applications, which may require up to 10¹⁵ particles for a single administration.

While the method is demonstrated in Sf9 insect cells, it is believed that other insect cells will provide similar results. Useful insects may include Anticarsia gemmatalis MNPV, Agrotis ipsilon nucleopolyhedrovirus, Autographa california MNPV, Bombyx mori NPV, Buzura suppressaria nucleopolyhedrovirus, Choristoneura fumiferana MNPV, Choristoneura fumiferana DEF nucleopolyhedrovirus, Choristoneura rosaceana nucleopolyhedrovirus, Culex nigripalpus nuclepoolyhedrovirus, Epiphyas postvitiana nucleopolyhedrovirus, Helicoverpa armisgera nucleopolyhedrovirus, Helicoverpa zea single nucleopolyhedrovirus, Lymantria dispar MNPV, Mamestra brassicae MNPV, Mamestra configurata nucleopolyhedrovirus, Neodiprion lecontii nucleopolyhedrovirus, Neodiprion sertifer NPV, Orgyia pseudotsugata MNPV, Spodoptera exigua MNPV, Spodoptera frugiperda MNPV, Spodoptera littoralis nucleopolyhedrovirus, Thysanoplusia orichalcea nucleopolyhedrovirus, Trichoplusia ni single nucleopolyhedrovirus, Wiseana signata nucleopolyhedrovirus.

Likewise the capsid protein may be selected from any one or more of the AAV serotypes, including AAV2, AAV4, AAV 5, AAV 6, AAV 7 and AAV 8. AAV 8 and AAV5 pseudotypes are particularly preferred because of :their known cell or tissue-targeting properties. SEQ ID NO.:3 is exemplary sequence of pseudotyped rAAV2/8 capsid.

Also contemplated as part of the invention are insect cells that harbor the recombinant insect virus vectors each encoding a small or large Rep protein and a Bac VP positioned tail-to-tail with the Rep sequence. The recombinant vectors may also include a chimeric AAV V1 protein partially substituted with an AAV phospholipid domain. A particularly preferred domain is AAV phospholipase A2 but other domains are expected to be useful.

Use of the terms “an”, “a” and “the” and similar terms used in claiming or describing the invention are intended to be construed as including both the singular and plural, unless clearly otherwise indicated or contraindicated. The terms “including”, “having” and “containing” are to be construed as open-ended in the same manner as the term “comprising” is commonly accepted as including but not limiting to the explicitly set forth subject matter. The term “comprising” and the like are constructed to encompass the phrases “consisting of” and “consisting essentially of.”

The methods and processes described herein may be performed in any suitable order unless otherwise indicated or clearly rendered inoperable by a modification in order.

Limited and narrow interpretation of descriptive language intended to better illustrate the invention is not to be construed as limiting in any way nor to limit the scope of the invention contemplated by the inventors.

### BRIEF DESCRIPTION OF THE FIGURES

FIG. 1 Western blot analysis of Rep proteins expressed in Sf9 cells by individual BacRep baculovirus helper plaque isolates, Isolate #5 (circled) was selected and propagated for the passage stability test (shown in FIG. 2).

FIG. 2 Western blot analysis of Rep proteins expressed in Sf9 cells by BacRep, BacRep52, or BacRep78 baculovirus helpers. Cells were infected with serially passaged baculovirus stocks (PI through P5) at MOI of 5.

FIG. 3 Western blot analysis of Rep proteins expressed in Sf9 cells by BacRep, BacRep52, or BacRep78 baculovirus helpers individually, or upon co-infection with other baculovirus helpers (MOI of 5 each). Lane 1—positive control (a lysate from 293 cells transfected with pIM45 (McCarty, et al, 1991); lanes 2 through 6 contain lysates from SIP cells infected with: lane 2—BacRep; lane 3—BacRep52; lane 4—BacRep78, lane 5—BacRep78+BacRep52; lane 6—BacRep78+BacRep52−BacVP+BacGFP (the latter vector also contains strong baculovirus p10 promoter driving GFP gene inside the transgene cassette (Urabe, et al., 2004)

FIG. 4 Passaging stability analysis of ITR-containing transgene cassette (BacGFP).

FIG. 4A—Analysis of rescued rAAV cassette. Sf9 cells were infected with BacGFP of consecutive passage stocks (MOI 5 each) in addition to BacRep (P2, MOI of 5). Forty eight hours post-infection, DNA was prepared by flirt DNA extraction, resolved using a 1.2% agarose gel, transferred to a Nylon filter and hybridized with a ³²P-labeled GFP probe.

FIG. 4B—Analysis of rAAV2-GFP titers of vector stocks prepared using BacGFP P2 through P5 helpers. Sf9 cells were co-infected with BacVP and BacRep (P2, MOI of 5 each). In addition, cells were co-infected with BacGFP at the indicated passages, (MOI 5 of each). Seventy-two hours post-infection, cells were harvested and rAAV infectious titers in crude cell lysates were calculated using GFP fluorescence assay using C12 cells co-infected with Ad5 (MOI of 10) (Zolotukhin, et al., 1999).

FIG. 5 Western blot analysis of AAV2 capsid proteins expressed in Sf9 cells by BacVP helper. Sf9 cells were infected with BacVP (MOI of 5) of consecutive passages, as indicated. Seventy-two hours post-infection, cells were harvested and cell lysates were analyzed by Western blotting as described.

FIG. 6 Silver stain polyacrylamide gel analysis of a fractionated iodixanol step gradient used to pre-purify rAAV2 prepared in Sf9 cells. The approximate positions of iodixanol density steps are shown above the upper edge of the gel. The mobility of rAAV capsid proteins VP1, VP2, and VP3 are indicated. Fractions containing full and empty particles are indicated.

FIG. 7 Analysis of the capsid protein VP I content and the respective VP 1 up phospholipase A2 activity in rAAV vector stocks produced in 293 cells vs. Sf9 cells.

FIG. 7A—Silver stain polyacrylamide gel analysis of purified rAA V stocks prepared in HEK 293 and Sf9 cells. The amounts of rAAV were normalized to contain approximately 10¹⁰ drp per lane. In the lane marked rAAVS/Sf9 five times more particles were loaded intentionally to show the low VP 1 content.

FIG. 7B—Thin layer chromatography of phospholipase A2 activity of virus produced in 293 cells vs. Sf9 cells. The same amounts of rAAV particles (approximately 10¹⁰ drp) as in A were analyzed by the assay as described in Materials and Methods. Lane 1 (positive control)—I ng of Bee Venom phospholipase (Sigma) was used.

FIG. 7C. Data from FIG. 7B quantified using phosphoimaging analysis. The lower phospholipase activity of rAAV2/293 vs. rAAV2/Sf9 reflected the lesser amount of particles added to the reaction (see FIG. 7A).

FIG. 8. Schematic representation of the AAV2 and AAV8 VP1 phospholipase domain swap.

FIG. 8A. Amino acid sequence alignment of VP1 up domains of AAV2 (SEQ ID NO: 1), AAV8 (SEQ ID NO: 2), and chimeric AAV2/8 (SEQ ID NO: 3).

FIG. 8B. Schematic drawing of the respective baculovirus vector cassettes expressing rAAV2, rAAV8, and rAAV2/8 capsids.

FIG. 9. Transduction of murine livers in vivo with rAAV8, or rAAV2/8. Mice were injected with 10¹² drp rAAV-GFP prepared from HEK 293 cells (rAAV8-GFP), Sf9 cells (rAAV8 GFP) or Sf9 cells (rAAV2/8)

FIG. 9A. HEK 293 cells (rAAV8-GFP)

FIG. 9B. Sf9 cells (rAAV8-GFP)

FIG. 9C. Sf9 cells (rAAV2/8). There was a robust GFP expression in hepatocytes except in rAAV8 prepared in Sf9 cells (FIG. 9B). Specificity of the GFP fluorescence was confirmed by the absence of fluorescence in the same field with a Rhodamine filter.

FIG. 10A. Physical map of pFBDLR(+) vector

FIG. 10B. Physical map of pFBDSR vector.

FIG. 11. Physical map of Baculovirus shuttle vector encoding AAV2/AAV8 capsid fusion protein.

### DETAILED DESCRIPTION OF THE INVENTION

The present invention was developed after analyzing the stability of the original baculovirus system components BacRep, BacVP, and transgene cassette-containing BacGFP.

In addressing the instability problem, a detailed analysis of the stability of the original baculovirus system components BacRep, BacVP and transgene cassette-containing BacGFP was undertaken. All the baculovirus helpers analyzed were prone to passaging-dependent loss-of-function deletions, resulting in considerable decreases in rAAV titers. To alleviate the instability problem, the Rep-encoding component was modified by splitting it into two separate vectors.

Additionally, the expression limits of the remaining components of the Baculovirus system were examined in order to optimize its application to AAV vector production. To successfully employ this system to pseudotyped AAV vectors, a novel modular approached of parvoviral phospholipase A2 (PLA2) domain swapping was introduced, allowing for baculovirus production of infectious AAV8 based vectors. The novel chimeric rAAV2/8 vector, produced in Sf9 cells, incorporated AAV2 PLA2 into AAV8 capsid structure and was characterized by robust transduction in vivo. This redesigned baculovirus system improved capacity for rAAV production and is applicable to other existing serotypes.

The expression limits of the remaining components of this system were examined in order to optimize its application to AAV vector production. To successfully employ this system with pseudotyped AAV vectors, a novel modular approach resulted in the discovery that parvoviral phospholipase A2 (PLA2) domain swapping can be used for baculovirus production of infectious AAV8 based vectors. The novel chimeric rAAV2/8 vector, produced in Sf9 cells, incorporates AAV2 PLA2 into an AAV8 capsid structure. This vector was tested and found to provide robust transduction in vivo. The novel redesigned baculovirus system improves the capacity for rAAV production and is applicable to other existing serotypes.

Pseudotyping. Pseudotyping is understood to mean that one or more structural proteins of a virus particle are not encoded by the viral nucleic acid. Generally, pseudotyped viruses include any recombinant viral gene transduction system that is dependent for genome packaging upon helper proteins expressed from defective genomes in viral producer cells or a “helper” virus. More particularly, a pseudotyped virus is understood to mean a virus in which the outer shell originates from a virus that differs form the source of the genome and the genome replication apparatus.

Current interest has focused on pseudotyped viral vectors in which the genome and outer shell come from different viruses; however, much work and interest have been directed to pseudotypes between different adeno-associated virus serotypes. The outer shell of the virus via interaction with cellular receptors has a major role in the tropism of the virus; i.e., at the entry level to the cell. Pseudotyping a viral vector can expand the number of target cells or, perhaps more desirably, restrict interaction to specific cell types. A pseudotyped vector can have an altered stability and/or interaction with the host immune system and may in some cases be concentrated to higher transduction titers than the “native” viral vector shell (Sanders, D. A., Current Opinion in Biotechnology 13: 437-442 (2002).

Tropism of AAV-2 has been effectively altered by pseudotyping the capsid from another serotype onto the AAV virion, which can alter cell binding and entry. The number of identified AAV serotypes at present is relatively small, but the differences achieved by capsid switching can be significant. So far, the serotype 8 capsid appears to show the most differences, especially in providing substantially improved liver transduction compared to. AAV-2. Cardiovascular tissue appears to be selectively transduced with pseudotyped AAV-6 virus, which contrasts with AAV-2, which localizes mainly in the liver after system administration. So far, AAV-3, AAV-4 and AAV-5 have yet to be associated with markedly changed tropism (Baker, Preclinica 2(6):November/December (2004).

Recombinant adeno-associated virus (rAAV) vectors have proved successful vehicles for delivery of a variety of genes. Currently, the most commonly tested and used AAV vector is constructed from AAV serotype 2, which is known to particularly target neurons in the CNS. Not all tissues are efficiently transduced with AAV2 vectors, so that even though delivery to different cell types occurs, high doses are needed to obtain therapeutically relevant levels of transgene expression. One approach to improving transduction is to package the AAV2 vector genome inside capsids from other AAV serotypes, of which several have been identified, including AAV1, AAV3, AAV4, AAV5, AAV6, AAV7 and AAV8. Vector pseudotypes have been prepared by packaging AAV2 genome in AAV6 or AAV8 capsids for example (Grimm, et al., Curr. Gene Ther. 3:281-304 (2003). Pseudotyped AAV6 was reported to successfully deliver genes to striated muscles (Gregorevic, et al., Nature Med. 10, 828-834 (2004).

The AAV5 capsid has generated particular interest because it is divergent from other capsid types, as indicated by detailed sequence comparisons with AAV2 and the other serotypes. The most divergent regions are thought to occur at the exterior surface of the mature virion (Bantel-Schaal, et al., J. Virol. 73:939-947 (1999); Hoshijima, M. et al. Nat. Med. 8, 864-871 (2002), which appears to account for the differences between AAV5 and AAV2 in cell targeting. Moreover, it has been suggested that AAV5 may utilize a different receptor and/or co-receptor for entering cells in such a manner as to enhance viral binding or endocytosis in certain cell types. This has been demonstrated in several different cell types, including airway epithelia and in pseudotyped rAAV2cap5 (Duan, et al., J. Virology 75, 7662-7671 (2001).

Numerous combinations of serotypes have been reported. Pseudotypes rAAV2/1, rAAV2/2 and rAAV2/5 engineered into vectors containing AAV2 terminal repeats flanking a GFP expression cassette under the control of a synthetic CBA promoter (Burger, et al., Mol. Ther. 10(2), 302-317 August (2004).

Other schemes have been used to target cells, including preparing AAV capsids that display immunoglobulin binding domains that target cell surface receptors (Ried, et al., 2002). An IgG binding domain of protein A, Z34Z, was inserted into the AAV2 capsid at amino acid position 587. The rAAV2-Z34C mutants coupled to antibodies against CD21 (B1-integrin), CD117(c-kit receptor) and CXCR4 were successful in transducing human hematopoietic cell lines.

Baculovirus Vectors. Baculoviruses are highly restricted insect viruses capable of entering a cell, but which cannot replicate in mammalian cells. Baculoviruses, unlike AAVs, can incorporate large amounts of extra genetic material, and express transgenes in mammalian cells when under the control of a mammalian or strong viral promoter. Gene delivery has been achieved in vitro and in vivo in dividing and non-dividing cells. The envelope protein gp64 can be mutated to develop targeted transduction of specific cell types Standbridge, et al., (2003). Over 500 strains of baculoviruses are recognized, including the subspecies Autographa californica multiple nuclear polyhedrosis viruses.

The invention, now described generally and in some detail, will be understood more readily by reference to the following examples, which are provided by way of reference and are in no manner intended to be limiting.

Materials and Methods

Spodoptera frugiperda Sf9 cells were grown at 27° C. in shaker flask cultures containing Sf-900 II SFM supplemented with 5% fetal bovine serum. All incubations for transfections and infections were done at 27° C.

Production of recombinant baculovirus Recombinant baculoviruses were constructed using Bac-to-Bac system (Gibco BRL). DH I OBac competent cells containing the baculovirus genome were transformed with the pFastBac transfer plasmids containing the AAV component insert: Bacmid DNA purified from recombination-positive white colonies was transfected into Sf9 cells using TransIT Insecta reagent (Mirus). Three days post-transfection, media containing baculovirus (pooled viral stock) was harvested and a plaque assay was conducted to prepare independent plaque isolates. Routinely, eight individual plaques were propagated to passage one (P1) to assay for the expression of the transgene or the ability of the transgene cassette to rescue and replicate as rAAV genome. Selected clones were propagated to P2, titered and used for large-scale rAAV preparations. Baculovirus titers were determined by plaque assay following the Bac-to-Bac system manual. Serial passaging was conducted as described by Kool, et al., Virology 192:94-101 (1993).

Production of rAAV vectors. Serum-free media-adapted Sf9 cells were used for large scale rAAV preparations. Sf9 cells at a density of 2-3×10′ cells/ml were co-infected with BacRep, BacVP, and BacGFP at multiplicity of infection (MOI) of 5 each, unless indicated otherwise. Alternatively, cells were co-infected with BacRep52 and BacRep78 (MOI of 5) to replace the BacRep virus. Three days post-infection, cells were harvested and processed as described earlier (Urabe, et al., Mol Ther 9:S160(2004)). Vectors were purified by iodixanol gradient centrifugation and column chromatography. Vectors were then concentrated and the buffer was exchanged in three cycles to Lactated Ringer's using Centrifugal Spin Concentrators (Apollo, 150 kDa cut-off, 20 ml capacity) (CLP). Physical and infectious rAAV particle titers were determined as described by Potter, et al., Methods Enzymol 346:413-30, (2002).

Western blot analysis. Sf9 cells (3×10⁶) were seeded in 6 cm dishes. Three days post infection, cells were harvested and lysed in 100 pL of buffer containing 50 mM Tris pH 7.6, 120 mM NaCl, 1 % Nonident P-40, 10% glycerol, 2 mM Na₃PO₄, 1 mM PMSF, 10 mM NaP₂O₇, 40 μg/mL leupeptin, 5 pg/mL aprotinin, 100 tM NaF, 1 mM EDTA, 1 mM EGTA, 1 μg/mL pepstatin. After incubation on ice for 1 hour, cell lysates were centrifuged at 12,000 rpm for 10 minutes. Clarified samples were separated by using SDS/10% polyacrylamide gel electrophoresis, transferred to a PVDF membrane and probed with the anti-AAV2 capsid monoclonal antibody B 1 (American Research Products) at 1:2000, which also recognizes the AAVI and AAVS capsid proteins (Wobus, et al., 2000, J Virol 74:9281-93), as well as AAV8 capsid, or with anti-Rep monoclonal antibodies (clone IF 11.8, 1:2000 dilution), depending on the context of the experiment. Detection was carried out using horseradish peroxidase (HRP)-conjugated sheep anti-mouse (Amersham Biosciences) at 1:5000 and ECL Western-Detection kit.

Phospholipase Assay. PLA mixed micelles assay was conducted as described previously (Zadori, et al, 2001, Dev Cell 1:291-302). Specifically, 10¹⁰ of purified DNAse I-resistant rAAV particles (drp) were pretreated for 2 min at 70° C. in 40 mM Tris pH 8.0 in a final volume of 17 μL. The assay was carried out in a total reaction volume of 50 μL containing the heat-treated virus in 100 mM TrisHCl, pH 8.0, 10 mM CaCl₂, 100 mM. NaCl, 1 mM Triton X-100, 40 μM phosphotidylcholine with 0.0625 pCi ¹⁴C-phosphotidylcholine. The reactions were incubated at 37° C. for 30 min. The products were extracted with chloroform:methanol:4M KCl (2:1:1). After centrifugation the products were separated by Silica gel thin layer chromatography with chloroform/methanol/water (65:35:4). The products were quantified by phosphoimaging analysis.

In vivo experiments. Animals were cared for in accordance with the principles of the Guide to the Care and Use of Experimental Animals. Vector (10¹² drp of rAAV8-GFP prepared in 293 or Sf9 cells, or rAAV2/8-GFP from Sf9 cells) was injected into the tail veins of C57BL/6 mice. Two weeks post-injection, mice were euthanized and tissues harvested for GFP visualization by direct fluorescence microscopy. Following fixation in 10% neutral buffered formalin overnight, samples were incubated in 30% sucrose in PBS (pH 7.4) for 24 hours then embedded in OCT media (Fisher). Cryosections (4 P m) were placed on slides and mounted (Vectashield with DAPI, Vector Labs, Calif.). Slides were viewed on a Zeiss Axioskop with a GFP filter (Chroma, 41028) and representative digital images taken from each animal at the same exposure settings using an Axiocam microscope. Autofluorescence was evaluated in the same field with a Rhodamine filter (Zeiss. Filter set 14, 510-560/590) and was negligible.

pFBDLR(+) and pFBDSR were constructed by subcloning the respective expression cassettes coding for large Rep78 and small; Rep52 from the pFBDLSR (Urabe, et al., 2002, Hum Gene-Ther 13:1935-43) into the pFastBacDual (Invitrogen) using standard molecular biology techniques.

DH10Bac competent E.coli cells were transformed with pFastBac containing either the Rep52 or Rep78 elements. Transformed clones were selected and bacmid DNA purified according to manual (Bac-to-Bac Baculovirus Expression Systems, GibcoBRL). Transfection of Sf9 cells was done with Mirus TransIT-Insecta transfection reagent according to the product manual. Four days after transfection media containing recombinant baculovirus was harvested. These stocks were subsequently plaque purified (O'Reilly, et al., Baculovirus expression vectors: a laboratory manual (1994)).

Sf9 (2.5×10⁶) cells were seeded in a 25 cm² flask and inoculated with 0.5 ml of the previous passage virus. After incubation for 2 hours, unabsorbed virus was aspirated and cells were washed twice with fresh media. The cells were incubated for 72 hours in 4 ml of media. This media was harvested and used to infect cells to produce the next passage virus. The virus used to produce the first passage of RepBac was the first generation amplified from a purified plaque. The virus used to produce the first passage of Rep78Bac and Rep52Bac were produced from transfection with respective bacmids.

Sf9 cells (3×10⁶) were seeded in 6 cm dishes in 3 ml of media and infected with 0.5 ml of undiluted virus produced by serial passaging. After 72 hours cells were harvested and lysed in 100 uL Sautin's Buffer (50 mM Tris pH 7.6, 120 mM NaCl, 1% Nonident P-40, 10% glycerol, 2 mM Na₃PO₄, 1 mM PMSF, 10 mM NaP₂O₇, 40 μg/uL leupeptin, 5 μg/uL aprotinin, 100 mM NaF, 1 mM EDTA, 1 mM EGTA, 1 μg/uL pepstatin). Lysed cells were incubated on ice for 1 hour and centrifuged at 12,000 rpm for 10 minutes. 50 μL supernatant was mixed with 25 μL 3× SDS running buffer. Samples were run on a 10% polyacrylamide gel for 6 hours at 275 volts, and transferred to a PVDF membrane. Primary antibody (IF11.5 anti-Rep monoclonal antibodies) were diluted 1:1000 and hybridized for 1 hr at room temperature. The blot was then incubated with a secondary antibody anti-mouse IgG labeled with horseradish peroxidase at a dilution of 1:1000. During antibody binding membranes were incubated in 1× PBS, 0.1% Tween 20, 5% milk. Before and after incubations membranes were washed 3 times for 10 minutes in 1× PBS, 0.1% Tween-20. Bands were visualized using chemiluminescent kit.

3×10⁶ cells were seeded on 6 cm dishes. One dish was infected with RepBac, VPBac, and GFPBac. Another dish was infected with Rep52Bac, Rep78Bac, VPBac, and GFPBac. All viruses were added at MOI 5. After 3 days, cells were harvested, lysed in 100 μL lysis buffer (150 mM NaCl, 50 mM Tris pH 8.5), subjected to three cycles of freeze-thaw, and centrifuged 12,000 rpm for 10 minutes. Serial dilutions of the supernatant were used to infect C12 cells in a 96 well plate. Adenovirus was also added at MOI 20. Two days after infection fluorescent cells were counted and infectious units per ml calculated. rAAV titer stock obtained with three baculoviral vectors (RepBac) was 1.9×10⁹ iu/ml, while stock obtained with four vectors (Rep52Bac+Rep78Bac) was 1.4×10⁹ iu/ml, which is essentially identical within experimental error.

### EXAMPLE 1

In order to provide comparison with other systems designed to increase rAAV production in competent host cells, the recombinant Baculoviruses reported by Urabe, et al., 2002, Hum Gene Ther 13:1935-43 were constructed.

Difficulties in scaling up rAAV production hinder the advancement of clinical protocols for gene therapy. Therefore, improvement in production methods, especially related to scale up, fulfills a need in the field. A recently developed baculovirus-based production protocol (Urabe, et al., 2002, Hum Gene Ther 13:1935-43), although potentially promising, was employed but produced only marginal titers. In addition, rAAV serotype 5 and 8 vectors, packaged using the baculovirus system disclosed in the reference, were non-infectious. The following procedures were used to investigate the cause of the baculovirus system instability and loss of Rep protein on sequential passaging.

pDG contains AAV rep and cap genes and E2A, E40RF6 and VA genes. According to the Urabe, et al, (2002), Hum Gene Ther 13:1935-43, the Rep52 to Rep78 ratio was increased by substituting the native p5 promoter with mouse mammary tumor virus (MMTV) long terminal repeat (LTR) promoter, a steroid-inducible promoter that is weakly active in noninduced conditions. The p19 promoter in the Rep ORF was reported to be constitutively active at much higher level than the MMTV LTR. To limit expression of Rep78 in Sf9 cells, the promoter for the immediate early 1 gene (IE-1) of Orgyia pseudotsugata nuclear polyhedrosis virus was used. The IE-1 promoter was partially deleted to limit expression of Rep78 even further (delta IE-1). The delta IE-1 promoter functioned at approximately 20% of the intact IE-1 promoter level (Theilmann and Stewart, 1991).

A three-vector system described by Kotin, et al., (US application 20040197895, 2004) was used to produce rAAV in Spodoptera frugiperda Sf9 cells grown at 27° C. in shaker flask cultures containing Sf-900II SFM supplemented with 10%FCS (WO 03/042361). Sf9 cells were infected with three recombinant baculoviruses; RepBac containing AAV2Rep78 and AAV2Rep52 expression cassettes; VPBac expressing AAV2 capsid proteins VP1, VP2 and VP3, and rAAV GFP marker transgene. While first passage production of rAAV using this 3-vector system was on the order of 5×10⁴ vector genomes/cell, Rep proteins failed to express on subsequent passages in Sf9 cells.

Western Blotting was employed to compare passaging results for the new constructs under the same conditions for the Rep vectors described by Urabe, et al. Hum Gene Ther 13:1935-43(2002). Data were obtained by comparing passaging results with separate and single Rep 52, Rep78 and Rep52/78 vectors in rAAV production studies.

In a typical experiment, cells were lysed in 1× sodium dodecyl sulfate (SDS) sample buffer and resolved on an SDS-Tris-glycine-10% polyacrylamide gel or a 4-12% NuPAGE Tris gel (Invitrogen). After electrophoresis, proteins were transferred to polyvinylidene difluoride (PVDF) membrane and incubated with a primary antibody, either an anti-Rep monoclonal antibody (303.9; Research Diagnostics, Flanders, N.J.) at a dilution of 1:200 or a polyclonal anti-VP antibody (Research Diagnostics) at a dilution of 1:2000. The blots were then incubated with a secondary anti-mouse or anti-rabbit immunoglobulin G labeled with horseradish peroxidase at a dilution of 1:7500 (Pierce, Milwaukee, Wis.). Membranes were incubated in TBS-T (10 mM Tris-HCl, pH 7.6, 0.15 M NaCl, 0.05% Tween 20). Antibodies were added to TBS-T for 1 hr. After incubation, membranes were washed three times for 10 min each in TBS-T. All steps were performed at ambient temperature.

### EXAMPLE 2

Stability of helper components. Upon re-plaquing the original BacRep stock, only 6 out of 10 individual plaque isolates expressed both Rep52 and Rep78, which was indicative of the inherent instability of the Rep helper construct. By splitting the palindromic orientation of the rep genes and designing two separate helpers expressing Rep52 and Rep78, the passaging stability of the vector was increased to P5. The re-designed set of vectors employed with a quadruple co-infection of Sf9 cells to produce rAAV appeared to provide improved results.

In a pilot experiment, side-by-side yields of rAAV prepared using three vs. four helpers (P2 each) at an MOI of 5 each were compared. There was little difference in rAAV titers produced (1.9×10⁹ infectious particles/ml-vs. 1.4×10⁹ infectious particles/ml).

In a separate experiment, whether or not an increased MOI of BacRep infection with a P3 stock would compensate for the partial loss of Rep-expressing baculovirus particles was tested. It was possible to compensate for such loss, or even to boost rAAV yield, by increasing the BacRep MOI to 15; i.e., in addition to two other baculovirus helpers, at an MOI of 5 each. An increase in rAAV titers when raising the MOI of individual helpers to 20, or combined MOI of 60 for the triple co-infection, was also noticed. Therefore, for rAAV production, there is a broad range of MOI that can be used with good results. This is a convenient feature because it permits a choice of additional protocols for infection with baculovirus helper combinations.

Two other components of the original helper set also appeared to be unstable during continuous passaging, with rAAV-ITR vector displaying a declining quality as early as P3. When propagating ITR-containing rAAV vector plasmids in E. coli, investigators conventionally utilize recombination pathway-deficient bacterial strains, such as SURE, to maintain the integrity of inverted terminal palindromic structures. No such strain appears to exist among insect cell lines. The stability of the ITR-containing helper after P2, therefore, appears to be a limiting factor for scaling up the system.

Even with such limitation, the total yield of P2 baculovirus vectors is sufficient to infect up to 300 L of Sf9 cells in suspension culture with an MOI of 5 to produce rAAV. Taking into account that even P3 helper vectors can be utilized at higher MOIs to compensate for the loss of the “active” helper component, the baculovirus system for rAAV production is believed to be robust enough for large-scale vector manufacturing.

### EXAMPLE 3

rAAV “pseudotyping”. The utility of the disclosed production system depends largely on the flexibility of its components to package (“pseudotype”) a particular rAAV cassette into other AAV serotype capsids. Vectors of other serotypes can achieve a higher transduction of a targeted tissue resulting in a reduced therapeutic vector dose.

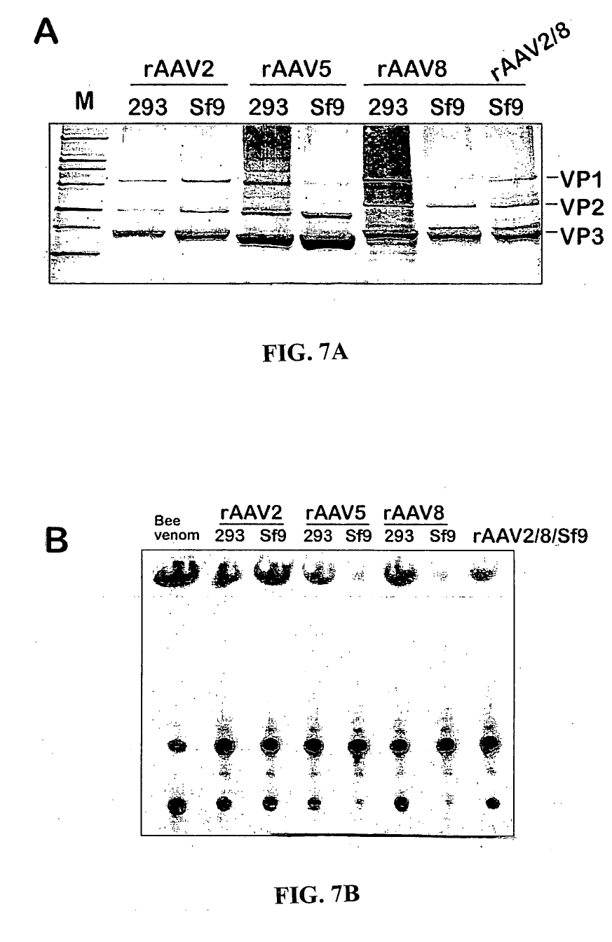

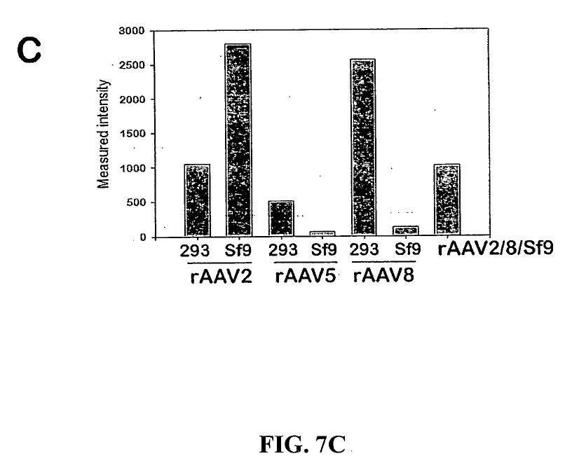

Initially, attempts to design BacVP-AAV5 and BacVP-AAV8 helper vectors by emulating the BacVP-AAV2 capsid helper were unsuccessful. Both rAAV serotype 5 and 8 (FIG. 7A) contained very little of VP1 known to harbor a phospholipase A2 domain that is critical for virus trafficking inside the cell. To alleviate the deficiency, the vector was redesigned by swapping the respective VP1up domains between AAV2 and AAV8 helpers. The resulting chimeric rAAV2/8 partially reconstituted the levels of VP1 protein and, as a result, increased PLA2 activity in vitro and infectivity in vivo.

### EXAMPLE 4

Using previously described procedures (Urabe, et al., Hum Gene Ther 13:1935-43 (2002)), rAAV2 vectors were produced by coinfecting insect Sf9 cells with three helper vectors: BacRep, BacVP, and BacGFP encoding rep, cap, and TR-embedded transgene cassette, respectively. Initial attempts to produce rAAV2 in this system resulted in titers that were significantly lower than reported. Consequently, the particular component(s) of the three baculovirus helpers responsible for the observed lower yields of rAAV2 were investigated.

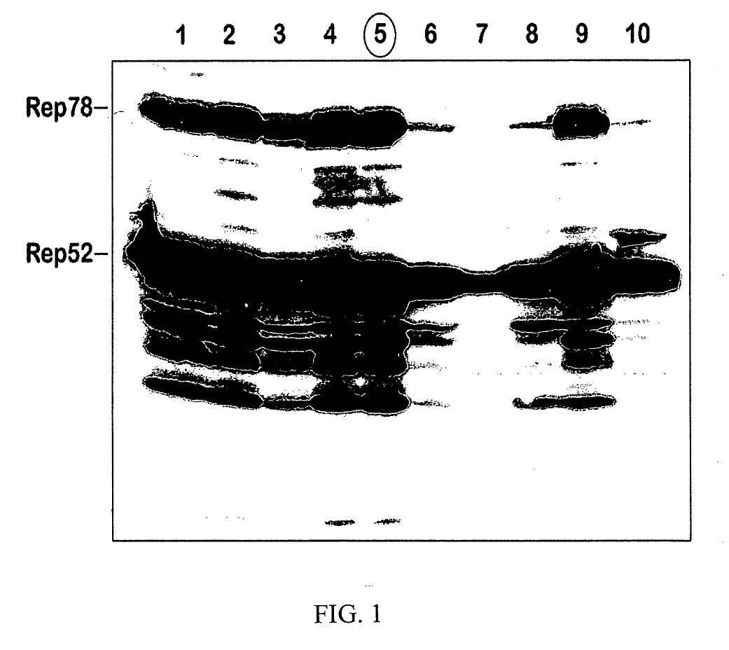

Rep component. Upon re-plaquing the. P3 BacRep, ten individual viral stocks of BacRep were amplified to generate P 1 stocks. For reference, the nomenclature describes the plaque itself as passage zero (P0), and the next generation of Baculovirus amplified from the plaque as P1. Sf9 cells were infected with P1 RepBacs and 3 days post-infection expression of Rep proteins was analyzed by Western blot. Four out of ten BacRep stocks produced relatively little Rep proteins in infected cells (FIG. 1). Titers of rAAV2 vector stocks produced using ten individual P1 isolates directly correlated with the amount of Rep proteins expressed by the individual helper. One stock was selected as the best producer among those tested (FIG. 1, lane 5), and was amplified and used in subsequent stability testing experiments.

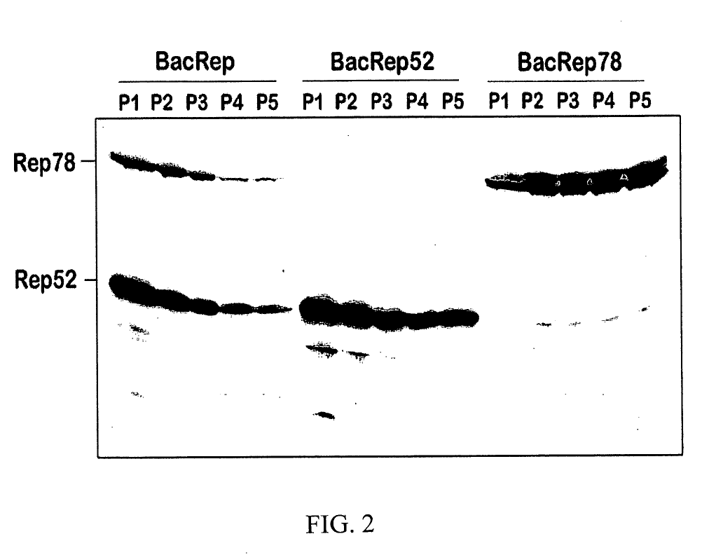

To determine the passaging stability of the selected BacRep, the helper virus was serially passaged up to P5, diluted to normalize for the gradual titer decrease as described by (Kool, et al., Virology 192:94-101(1993)) and the expression of Rep proteins was analyzed by Western blot (FIG. 2, panel BacRep). The expression of both Rep78 and Rep52 in BacRep-infected cells declined with each passage.

### EXAMPLE 5

In a previously described BacRep helper (Urabe, et al., Hum Gene Ther 13:1935-43(2002)), AIE1-driven rep 78 and pohl-driven rep52 were placed in a head-to-head orientation creating, in effect, a perfect palindrome structure of about 1.2 Kbp. In the wtAAV genome, these two genes are encoded by two collinear ORFs within one DNA sequence, transcribed into two separate mRNAs from the P5 and P19 promoters. It was hypothesized that in the helper, the palindrome orientation of rep52 and rep78 sequences within the baculovirus genome could result in the formation of an unstable secondary structure leading to recombination and subsequent deletion during replication.

To test this hypothesis, the rep52 and rep 78 genes were sub-cloned to derive two separate recombinant baculoviruses, BacRep52 and BacRep78 that retained the original expression cassettes, including promoters. Individual vector stocks, prepared as described above, were analyzed for the production of Rep52 and Rep78 proteins. The best producers were, selected, serially passaged to derive P5, and Rep expression levels were visualized by Western blot. Unlike the BacRep described by Urabe, et al., levels of Rep proteins remained either constant (Rep78) or declined only slightly (Rep52) from the first passage stock to the fifth (FIG. 2, panels BacRep52 and BacRep78). In this experiment, when expressed separately, AIE1-driven rep78 and pohl-driven rep52 produced comparable amounts of Rep proteins. In addition, BacRep78 produced small amounts of Rep52 derived from mRNA transcribed from AAV2 P19 promoter, suggesting the viral P19 sequence retains some residual promoter activity in insect cells.

### EXAMPLE 6

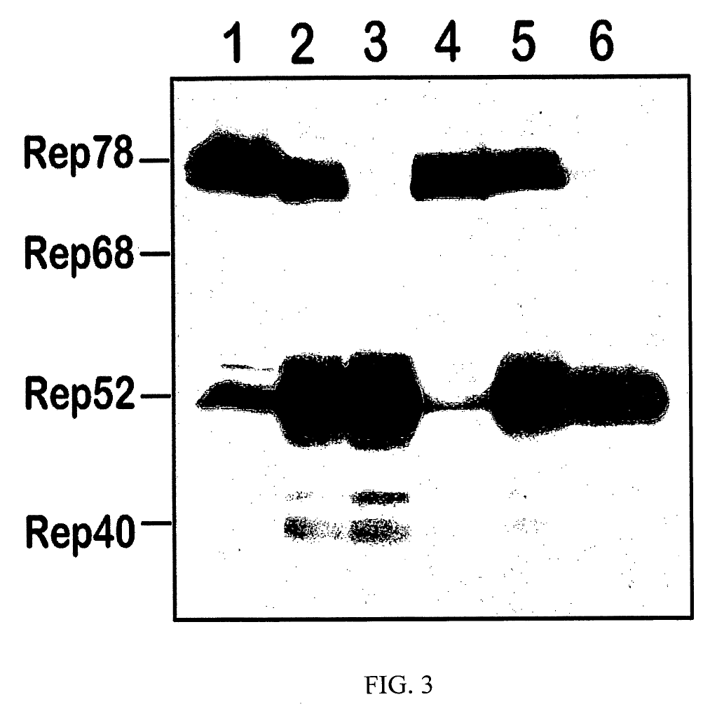

The high stoichiometric ratio of Rep52/Rep78 in favor of the former is recognized as a factor in obtaining a high yield of rAAV (Xiao, et al., Virol 72:2224-32 (1998)). This example was addressed to whether or not Rep stoichiometry changes under the conditions of quadruple co-infection with these helper viruses. Seventy-two hours post infection with various combinations of helper vectors (MOI. of 5 each), Rep proteins were analyzed by Western blotting analysis (FIG. 3). Infection with BacRep78, or BacRep52 alone produced ratios, which were similar to the original BacRep construct (FIG. 3, lanes 2-4). However, this ratio was shifted slightly in favor of Rep52 (FIG. 3, lane 5) when cells were co-infected with both BacRep78 and BacRep52. Moreover, when two additional baculovirus promoters were introduced (pohl in BacVP and p10 in BacGFP in a quadruple co-infection), this ratio shifted in favor of the small Rep (FIG. 3, lane 6), suggesting that three strong viral promoters may compete for available transcription factors and attenuated the AIE1 promoter.

### EXAMPLE 7

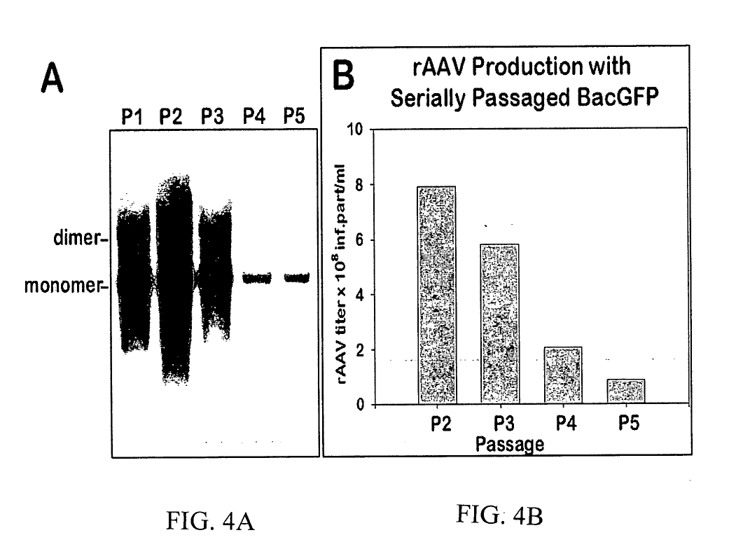

AAV2 ITR-flanked transgene cassette component. The palindromic termini of the AAV genome, as well as rAAV derivatives are notoriously unstable and prone to deletions that render the genome functionally defective. This example was designed to answer whether the ITR-containing component of the helper triumvirate would maintain functional replicative capability for the duration of five consecutive passages. There was a notable loss of the ITR-transgene cassette-containing baculovirus over the 5 passages. This reduction was documented by assaying rescued TR-containing cassette replicating in the presence of Rep proteins (FIG. 4A). Titers of rAAV2-GFP, prepared using the respective P1 through P5 BacGFP helpers (MOI of 5 each) closely correlated with the reduction of the ITR-containing sequences (FIG. 4B).

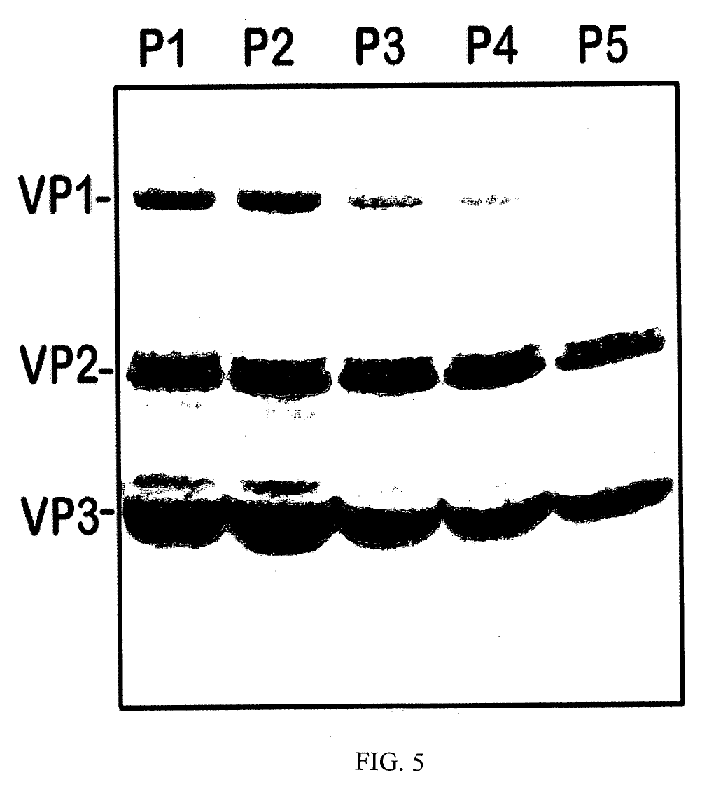

VP component. Similarly, as in Example 7, the five-passage stability test was applied to the original BacVP viral stock component. As with the other components of this production system, Western blotting analysis demonstrated a notable decline in VP1, VP2, and VP3 capsid proteins expressed by helper vectors from the P1 to P5 (FIG. 5).

### EXAMPLE 8

The overall utility of the baculovirus AAV production system ultimately resides on its ability to “pseudotype” an AAV2-ITR transgene cassette with capsid genes of other AAV serotypes. BacVP helper vectors were designed to produce AAV5 and AAV8 pseudotyped rAAVs. The constructs were designed to emulate the pFBDVPml 1 construct described by Urabe, et al. (2002)) introducing similar mutations into-AAV5 and AAV8 capsid genes encoding VP1 N-termini. Eight individual plaques of each construct were screened to identify BacVP5 and BacVP8. helper vectors using Western blotting analysis; selected clones were propagated to P2 and used in triple co-infection with BacRep and BacGFP to produce pseudotyped rAAV5-GFP and rAAV8-GFP.

Titers of the purified rAAV5 and rAAV8 stocks were similar to rAAV2 titers approaching 5×10⁴ drp per cell. However, in contrast to rAAV2-GFP, the particle-to-infectivity ratios of rAAV5-GFP and rAAV8-GFP were generally by 3-4 orders of magnitude higher (as assayed on HeLa-derived C12 cells upon Ad5 co-infection). The reason for the extremely low infectivity of Sf9-derived serotype 5 and 8 vectors was revealed upon closer investigation of the capsid composition in purified viral particles.

Iodixanol gradients have been reported as effective for the purification of rAAV2 produced in 293 cells (Zolotukhin, et al., Gene Ther 6:973-85 (1999)). Furthermore, these iodixanol gradients are capable of separating full from empty AAV particles (Potter, et al., Methods Enzymol 346:413-30 (2002)).

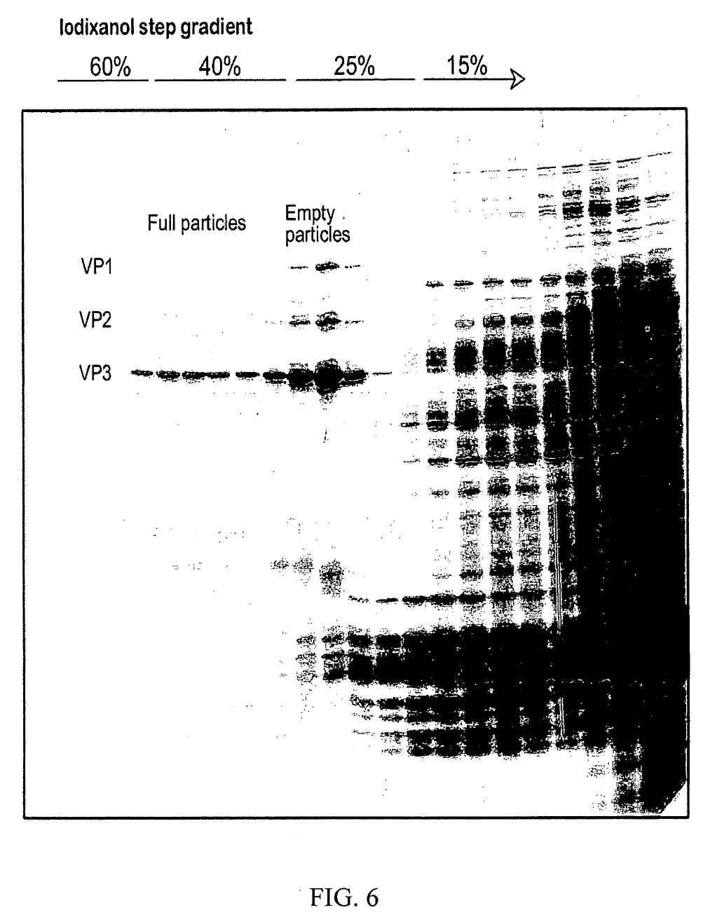

This technique was used to pre-purify rAAV produced in Sf9 cells to analyze the capsid stoichiometry of the fully assembled DNA-containing particles. FIG. 6 demonstrates typical SDS-PAGE gel analysis of fractionated iodixanol gradient from Sf9 cell lysate containing rAAV2-GFP. rAAVS and rAAV8, pre-purified in a similar fashion, were further purified using Q Sepharose anion-exchange chromatography and concentrated. The concentrated rAAV stocks were analyzed using SDS-PAGE and silver staining analysis (FIG. 7A). The capsid protein compositions of both 293- and Sf9-derived rAAV2 capsids were similar, with VPI:VP2:VP3 ratios approximating 1:1:10. However, the amounts of VPI in Sf9-derived rAAV5 and 8 were considerably lower as compared to their 293 counterparts.

### EXAMPLE 9

Girod, et al., J. Gen. Virology, 83:975-8 (2002); Wobus, et al., J. Virol., 74:9281-93 (2002) have shown that the N-terminus of the AAV VP1 capsid protein contains a phospholipase A2 (PLA2) motif that is critical for efficient viral infection. Mutations in this VP1 unique region had no influence on capsid assembly, packaging of viral genomes or binding to and entry into cells. However, this PLA2 activity is required for endosome exit and viral genome transfer into the nucleus (Zadori, et al., Dev Cell 1:291-302(2001)). The data showed that the BacVP-AAV5 and BacVP-AAV8 helpers did not provide sufficient VP1 for a fully infectious viral particle. To determine whether the shortage of VP1 and, ultimately, low PLA2 activity of the “pseudotyped”, capsids is responsible for the observed infectious titers of these serotypes produced in Sf9 cells, in vitro phospholipase assays were conducted using purified vector preparations (FIG. 7B, C). Indeed, while AAV2 prepared in both 293- and Sf9 cells displayed comparable PLA2 activity that correlated with their respective particle-to-infectivity ratios, both AAV5-GFP and AAV8GFP had significantly lower PLA2 activity when produced in Sf9 cells.

PLA2 domain swapping. Urabe et al., Hum Gene Ther 13:1935-43 (2002)) have modified the N-terminus of the VP1 ORF. The introduced mutations provided the proper stoichiometry for the capsid proteins and for the assembly of infectious rAAV vector produced in insect cells, which were indistinguishable from 293-derived virus. An attempt to use this same approach for the production of pseudotyped AAV5 and AAV8 vectors by introducing similar mutations resulted in assembly of non-infectious viral particles.

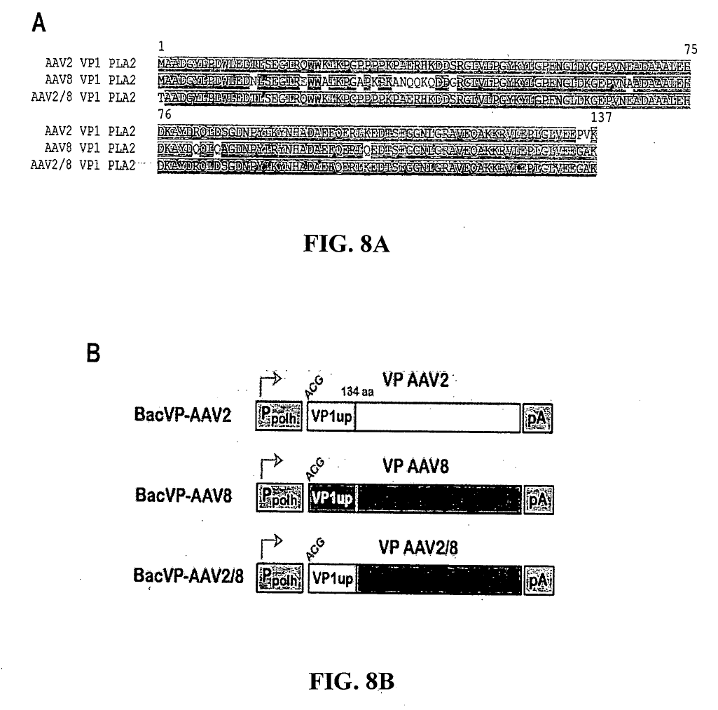

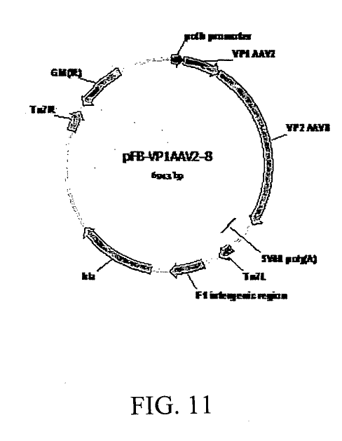

It was hypothesized that swapping the portion of the capsid ORF encoding the AAV2 PLA2 domain for the homologous sequence in BacVP-AAV8 might improve the capsid protein stoichiometry in the resultant particles. To this end, the 134 N-terminal amino acid residues of AAV2 VP1 were substituted for the respective domain in AAV8 VPI (FIG. 8) using a PCR-mediated protocol. Upon sequence verification, the chimeric BacVP-AAV2/8 helper vector was constructed (FIG. 11) and a viral stock propagated. The particle titers of rAAV2/ 8-GFP prepared using this chimeric helper were similar to rAAV2, 5, or 8 serotypes produced in Sf9 cells. After purification using the iodixanol/Q-Sepharose protocol, the capsid composition was analyzed by SDS-protein gel electrophoresis (FIG. 7A, last lane). The amount of AAV2/8 VP1 present within the particle was increased, although the level of this chimeric VP1 was not equivalent to AAV8 VP2. Yet, the PLA2 assay confirmed this partial recovery was sufficient to increase the particles phospholipase activity supporting the original hypothesis (FIG. 7B, C).

### EXAMPLE 10

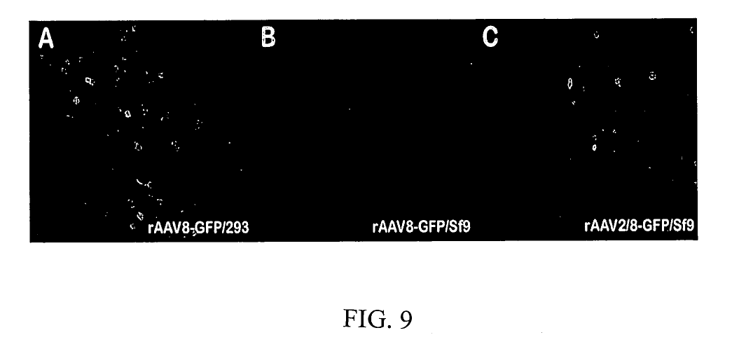

Transduction of murine tissues in vivo. To test transduction efficiencies of the baculovirus-derived rAAV vectors, 10¹² particles of rAAV-GFP preparations were injected into the tail vein of adult mice, using rAAV8-GFP produced in 293 cells as a positive control. Three weeks post-injection, animals were euthanized, tissues were harvested, and transduction was visually estimated by the intensity of direct GFP fluorescence. All the analyzed tissues, including liver, cardiac muscle, pancreas, spleen, and lung were robustly transduced with rAAV8-GFP prepared in 293 cells (for the purpose of clarity, in FIG. 9A only transduction of liver is shown). On the contrary, rAAV8-GFP derived from Sf9 cells, was essentially non-infectious (FIG. 9B). At the same time, rAAV2/8-GFP (also Sf9 cells-derived) demonstrated high transduction efficiencies in liver comparable to the vector derived from mammalian cells (FIG. 9C). This resulted in a chimeric rAAV2/8 vector that was highly infectious in vivo.

### EXAMPLE 11

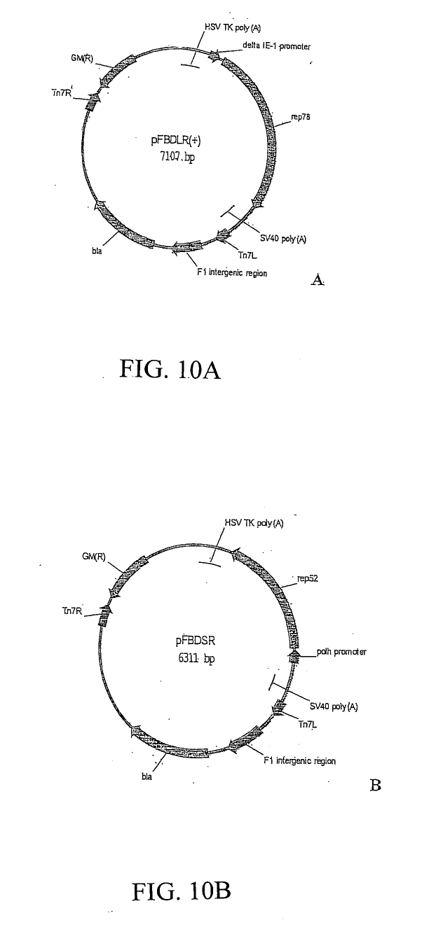

Two redesigned recombinant baculovirus vectors encoding Rep52 and Rep78 were constructed. Vector pFBDLR(+) is shown in FIG. 10A and vector pFBDSR in FIG. 10B. A Bac52/78 vector was prepared using a standard procedure similar to the standard procedure described in Example 1. Separate baculovirus vectors, Bac52 and Bac78 were prepared using similar standard procedures as outlined in Example 2. The procedures for virus production and passaging were used as set forth in Example 2. After each passage, the amount of Rep protein produced in the lysed cell was determined by Western Blot analysis. Results showed a significant difference in procedures using separate rep52 and rep78 Baculovirus vectors.

A Western blot analysis of Rep proteins from lysed Sf9 cells infected with recombinant Bac52/78Rep showed decreased expression of Rep 78 from Bac52/78Rep with multiple passaging and virtually no protein after 5 passages. Rep 52 showed a similar loss with only a fraction of the Rep52 protein observed after 5 passages. In contrast, Bac52Rep and Bac78Rep continued to exhibit vigorous expression after 5 passages, indicating little, if any, loss.

Discussion of Results

rAAV2 vectors were produced in accordance with the procedures described by Urabe, et al. (2002) by coinfecting insect Sf9 cells with three helper vectors: BacRep, BacVP, and BacGFP encoding rep, cap, and TR-embedded transgene cassette, respectively. An initial attempt to produce rAAV2 in this system resulted in titers that were significantly lower than reported by the authors. Consequently, efforts were directed to determining which particular component(s) of the three baculovirus helpers were responsible for the observed lower yields of rAAV2.

Rep component. Upon re-plaquing the P3 BacRep, ten individual viral stocks of BacRep were amplified to generate P1 stocks. For reference, the nomenclature describes the plaque itself as passage zero (PO), and the next generation of baculovirus amplified from the plaque as P1. Sf9 cells were infected with P1 RepBacs and 3 days post-infection expression of Rep proteins was analyzed by Western blot. Four out of ten BacRep stocks produced relatively little Rep proteins in infected cells (FIG. 1). Titers of rAAV2 vector stocks produced using ten individual P1 isolates directly correlated with the amount of Rep proteins expressed by the individual helper. One stock was selected as the best producer among those tested (FIG. 1, lane 5), and was amplified and used in subsequent stability testing experiments.

To determine the passaging stability of the selected BacRep, the helper virus was serially passaged up to P5, diluted to normalize for the gradual titer decrease as described by Kool et al. Virology 192:94-101, (1993) and the expression of Rep proteins was analyzed by Western blot (FIG. 2, panel BacRep). The expression of both Rep78 and Rep52 in BacRep-infected cells declined with each passage.

In the original BacRep helper of Urabe, et al. (2002), OIE1-driven rep78 and polh-driven rep52 were placed in a head-to-head orientation creating, in effect, a perfect palindrome structure of about 1.2 Kbp. In the wtAAV genome, these two genes are encoded by two collinear ORFs within one DNA sequence, transcribed into two separate mRNAs from the P5 and P19 promoters. It seemed possible that in the helper, the palindrome orientation of rep52 and rep78 sequences within the baculovirus genome could result in the formation of an unstable secondary structure leading to recombination and subsequent deletion during replication. To test this hypothesis, the rep52 and rep 78 genes were sub-cloned to derive two separate recombinant baculoviruses, BacRep52 and BacRep78 that retained the original expression cassettes, including promoters.

Individual vector stocks, prepared as described above, were analyzed for the production of Rep52 and Rep78 proteins, the best producers selected, serially passaged to derive P5, and Rep expression levels visualized by Western blot. Unlike the original BacRep, levels of Rep proteins appeared to remain either constant (Rep78) or declined only slightly (Rep52) from the first passage stock to the fifth (FIG. 2, panels BacRep52 and BacRep78). In this experiment, when expressed separately, AIE1-driven rep78 and polh-driven rep52 produced comparable amounts of Rep proteins. In addition, BacRep78 produced small amounts of Rep52 derived from mRNA transcribed from AAV2 P19 promoter, suggesting that the viral P19 sequence retains some residual promoter activity in insect cells.

The high stoichiometric ratio of Rep52/Rep78 in favor of the former is known to be an important factor for the high yield of rAAV (Xiao, et al., J. Virol 72:2223-32 (1998)). The next question was whether or not Rep stoichiometry changes under the conditions of quadruple co-infection with these helper viruses.

Seventy-two hours post infection with various combinations of helper vectors (M.O.I. of 5 each), Rep proteins were analyzed by Western blotting analysis (FIG. 3). Infection with BacRep78, or BacRep52 alone produced ratios, which were similar to the original BacRep construct (FIG. 3, lanes 2-4). However, this ratio was shifted slightly in favor of Rep52 (FIG. 3, lane 5) when cells were co-infected with both BacRep78 and BacRep52. Moreover, when two additional baculovirus promoters were introduced (polh in BacVP and p10 in BacGFP in a quadruple co-infection), this ratio shifted in favor of the small Rep (FIG. 3, lane 6), suggesting that three strong viral promoters may compete for available transcription factors and attenuated the AIE1 promoter.

AAV2 ITR-flanked transgene cassette component. The palindromic termini of the AAV genome, as well as rAAV derivatives are notoriously unstable and prone to deletions that render the genome functionally defective. Another experiment was designed to determine whether or not the ITR-containing component of the helper triumvirate would maintain functional replicative capability for the duration of five consecutive passages. There was a notable loss of the ITR-transgene cassette-containing baculovirus over the 5 passages. This reduction was documented by assaying rescued TR-containing cassette replicating in the presence of Rep proteins (FIG. 4A). Titers of rAAV2-GFP, prepared using the respective P1 through P5 BacGFP helpers (MOI of 5 each) closely correlated with the reduction of the ITR-containing sequences (FIG. 4B).

VP component. Similarly, the five-passage stability test was applied to the original BacVP viral stock component. As with the other components of this production system, Western blotting analysis demonstrated a notable decline in VP1, VP2, and VP3 capsid proteins expressed by helper vectors from the P1 to P5 (FIG. 5).

The overall utility of the baculovirus AAV production system ultimately resides on its ability to “pseudotype” an AAV2-ITR transgene cassette with capsid genes of other AAV serotypes. Therefore BacVP helper vectors were designed to produce AAV5 and AAV8 pseudotyped rAAVs. The constructs were designed to emulate the pFBDVPml 1 construct described by Urabe, et al. (2002) by introducing similar mutations into AAV5 and AAV8 capsid genes encoding VPI N-termini. Eight individual plaques of each construct were screened to identify BacVP5 and BacVP8 helper vectors using Western blotting analysis; selected clones were propagated to P2 and used in triple co-infection with BacRep and BacGFP to produce pseudotyped rAAV5-GFP and rAAV8-GFP.

Titers of the purified rAAV5 and rAAVS stocks were similar to rAAV2 titers approaching 5×10 drp per cell. However, in contrast to rAAV2-GFP, the particle-to infectivity ratios of rAAV5-GFP and rAAV8-GFP were generally 3-4 orders of magnitude higher (as assayed on HeLa-derived C 12 cells upon Ad5 co-infection). The reason for the extremely low infectivity of SO-derived serotype 5 and 8 vectors was revealed upon closer investigation of the capsid composition in purified viral particles.

It was previously reported that iodixanol gradients are effective for the purification of rAAV2 produced in 293 cells (Zolotukhin, et al., 1999). Furthermore, these iodixanol gradients are capable of separating full from empty AAV particles. This technique was employed to pre-purify rAAV produced in Sf9 cells to analyze the capsid stoichiometry of the fully assembled DNA-containing particles. FIG. 6 demonstrates typical SDS-PAGE gel analysis of fractionated iodixanol gradient from Sf9 cell lysate containing rAAV2-GFP. rAAV5 and rAAV8, pre-purified in a similar fashion, were further purified using QSepharose anion-exchange chromatography and concentrated. The concentrated rAAV stocks were analyzed using SDS-PAGE and silver staining analysis (FIG. 7A). The capsid protein compositions of both 293- and Sf9-derived rAAV2 capsids were similar, with VP 1:VP2:VP3 ratios approximating 1:1:10. However, the amounts of VPI in Sf9-derived rAAV5 and 8 were considerably lower as compared to their 293 counterparts.

Girod et al., J. Gen. Virol 83:973-8 (2002) have shown that the N-terminus of the AAV VP 1 capsid protein contains a phospholipase A2 (PLA2) motif that is critical for efficient viral infection. Mutations in this VPI unique region had no influence on capsid assembly, packaging of viral genomes or binding to and entry into cells. However, this PLA2 activity is required for endosome exit and viral genome transfer into the nucleus. Therefore, it appeared from the data that the BacVP-AAV5 and BacVP-AAV8 helpers did not provide sufficient VP1 for a fully infectious viral particle. To test whether or not the shortage of VP1 and, ultimately, low PLA2 activity of the “pseudotyped” capsids is responsible for the observed infectious titers of these serotypes produced in Sf9 cells, in vitro phospholipase assays were conducted using purified vector preparations (FIG. 7B, C). Indeed, while AAV2 prepared in both 293- and Sf9 cells displayed comparable PLA2 activity that correlated with their respective particle-to-infectivity ratios, both AAV5-GFP and AAV8GFP had significantly lower PLA² activity when produced in Sf9 cells.

PLA2 domain swapping In order to produce AAV2 in insect cells, Urabe et al (2002) modified the N-terminus of the VPI ORE. The introduced mutations allowed for the proper stoichiometry of the capsid proteins and for the assembly of infectious vector indistinguishable from 293-derived virus. On the other hand, an initial attempt to emulate this approach for the production of pseudotyped AAV5 and AAV8 vectors by introducing similar mutations resulted in assembly of non-infectious viral particles.

It was therefore hypothesized that swapping the portion of the capsid ORF encoding the AAV2 PLA2 domain for the homologous sequence in BacVP-AAV8 might improve the capsid protein stoichiometry in the resultant particles. To this end, 134 N-terminal amino acid residues of AAV2 VPI were substituted for the respective domain in AAV8 VP1 (FIG. 8) using a PCR-mediated protocol. Upon sequence verification, the chimeric BacVP-AAV2/8 helper vector was constructed and a viral stock propagated. The particle titers of rAAV2/8-GFP prepared using this chimeric helper were similar to rAAV2, 5, or 8 serotypes produced in Sf9 cells. After purification using the iodixanol/QSepharose protocol, the capsid composition was analyzed by SDS-protein gel electrophoresis (FIG. 7A, last lane). As anticipated, the amount of AAV2/8 VP1 present within the particle was increased, although the level of this chimeric VP 1 was not equivalent to AAV8 VP2. Yet, the PLA2 assay confirmed that this partial recovery was sufficient to increase the particles phospholipase activity supporting the original hypothesis (FIG. 7B, C).

Transduction of murine tissues in vivo. To test transduction efficiencies of the baculovirus-derived rAAV vectors, 10¹² particles of rAAV-GFP preparations were injected into the tail vein of adult mice, using rAAV8-GFP produced in 293 cells as a positive control. Three weeks post-injection, animals were euthanized, tissues were harvested, and transduction was visually estimated by the intensity of direct GFP fluorescence. All the analyzed tissues, including liver, cardiac muscle, pancreas, spleen, and lung were robustly transduced with rAAV8-GFP prepared in 293 cells. For the purpose of clarity, in FIG. 9A only transduction of liver is shown. On the contrary, rAAV8-GFP derived from Sf9 cells, was essentially non-infectious (FIG. 9B). At the same time, rAAV218-GFP (also Sf9 cells-derived) demonstrated high transduction efficiencies in liver comparable to the vector derived from mammalian cells (FIG. 9C). The results were a chimeric rAAV2/8 vector that was highly infectious in vivo.

Difficulties in scaling up the rAAV production hinder the advancement of clinical protocols for gene therapy. Therefore, every improvement in production methods, especially related to up-scaling, is welcome in the field. The recently developed Baculovirus-based production protocol of Urabe, et al., was followed, but produced only marginal titers. In addition, rAAV serotype 5 and 8 vectors, designed and packaged using baculovirus system, were non-infectious. The cause of the Baculovirus system variability was determined by testing the stability of each individual component of the system.

Stability of helper components. Upon re-plaquing the original BacRep stock, only 6 out of 10 individual plaque isolates expressed both Rep52 and Rep78, which was indicative of the inherent instability of the Rep helper construct. By splitting the palindromic orientation of the rep genes and designing two separate helpers expressing Rep52 and Rep78, the passaging stability of the vector was increased to at least P5. Use of the re-designed set of vectors showed that a quadruple instead of triple co-infection of Sf9 cells to produce rAAV avoided loss of Rep on multiple passaging. In a pilot experiment, side-by-side yields of rAAV prepared using three vs. four helpers (P2 each) at an MOI of 5 each were compared.

There was little difference in rAAV titers produced (1.9×10⁹ inf. part./ml vs. 1.4×10⁹ inf.part./ml). In a separate experiment, it was determined whether or not the increased MOI of BacRep infection with a P3 stock would compensate for the partial loss of Rep-expressing baculovirus particles (FIG. 10A). It appears that it is indeed possible to compensate for such loss, or even to boost rAAV yield by increasing the BacRep MOI to 15 (that is, in addition to two other baculovirus helpers, MOI of 5 each). Curiously, an increase in rAAV titers was also noticed when raising the MOI of individual helpers to 20 (or combined MOI of 60 for the triple co-infection) (FIG. 10B). Therefore, for rAAV production, there seems to be a broad range of MOI allowing for more flexible infection of baculovirus helper combinations.

Two other components of the original helper set also appeared to be unstable during continuous passaging, with rAAV-ITR vector displaying a declining quality as early as at P3. When propagating ITR-containing rAAV vector plasmids in E.coli, investigators conventionally utilize recombination pathways-deficient bacterial strains, such as SURE to maintain the integrity of inverted terminal palindromic structures. Such an equivalent does not appear to exist among insect cell lines. The stability of the ITR-containing helper after P2, therefore, appears to be a limiting factor for scaling up the system.

However, even with such limitation, the total yield of P2 baculovirus vectors is sufficient to infect up to 300 L of Sf9 cells in suspension culture with an MOI of 5 to produce rAAV. Taking into account that even P3 helper vectors can be utilized at higher MOIs to compensate for the loss of the “active” helper component, the baculovirus system for rAAV production appears to be robust enough for large-scale vector manufacturing.

Using baculovirus system for rAAV “pseudotyping The utility of the production system depends largely on the flexibility of its components to package (“pseudotype”) a particular rAAV cassette into other AAV serotype capsids. Vectors of other serotypes can achieve a higher transduction of a targeted tissue resulting in a reduced therapeutic vector dose. Initially, by designing BacVP-AAV5 and BacVP-AAV8 helper vectors that emulated the BacVP-AAV2 capsid helper, results were discouraging. Both rAAV serotype 5 and 8 (FIG. 7A) contained very little of VP1 known to harbor a phospholipase A2 domain that is critical for virus trafficking inside the cell. To alleviate the deficiency, the vector was redesigned by swapping the respective VP1 up domains between AAV2 and AAV8 helpers. The resulting chimeric rAAV2/8 partially reconstituted the levels of VP1 protein and, as a result, increased PLA2 activity in vitro and infectivity in vivo.

In in vivo experiments, the re-designed chimeric rAAV2/8-GFP appeared to be targeted mainly to the liver, unlike to the mammalian cell-derived rAAV8-GFP, which transduced indiscriminately all the tissues tested. The hepatocyte-specific transduction may have resulted from the overall reduced VP1 PLA2 activity, or from the change in vector tropism.

The described work further extends the agility of AAV vector system by demonstrating that VP1 up domains of the AAV viruses are completely modular and can be replaced with homologous domains from other parvoviral capsids, or even with completely unrelated phospholipases such as bee venom PLA or porcine parvovirus PLA. It is contemplated that such interchangeable PLA modules may be utilized as universal building blocks for a novel, highly efficacious vector platform combining serotype tropism diversity with superior transduction rates. The re-designed baculovirus system disclosed herein enhances the capacity for rAAV production making the AAV platform more amenable to large-scale clinical manufacturing.

The methods, techniques and compositions disclosed and claimed herein can be made and executed without undue experimentation in light of the present disclosure. While the compositions and methods of this invention have been illustrated with several examples and preferred embodiments, it will be apparent to those of skill in the art that variations may be applied to the methods and compositions, in the steps or in the sequence of steps and in modifications of the compositions without departing from the concept, spirit and scope of the invention. Accordingly, the exclusive rights sought to be patented are as described in the claims below.

### REFERENCES

All references, including patents, published patent applications, scientific publications and publically available material cited herein are hereby incorporated by reference to the same extent as if each reference were individually and specifically set forth as being specifically incorporated by reference in its entirety herein.
Baker, A. H., “Adeno-associated viral vectors for cardiovascular gene therapy: current perspectives”, Preclinica 2(6): November/December (2004).
Bantel-Schaal, U., Delius, H., Schmidt, R. and zur Hausen, H. “Human adeno-associated virus type 5 is only distantly related to other known primate helper-dependent parvoviruses”, J. Virol. 73:939-947 (1999).
Burger, C., Gorbatyuk, O. S., Velardo, M. J., Peden, C. S., Williams, P., Zolotukhin, S., Reier, P. J., Mandel, R. J. and Muzyczka, N. “Recombinant AAV viral pseudotyped with viral capsids from serotypes 1,2,and 5 display differential efficiency and cell tropism after delivery to different regions of the central nervous system” Mol. Ther. 10(2), August 2004, pp. 302-317.
Duan, et al., “Enhancement of muscle gene delivery with pseudotyped adeno-associated virus type 5 correlates with myoblast differentiation” J. Virology 75, 7662-7671 (2001).
Gao, G., M. R. Alvira, S. Somanathan, Y. Lu, L. H. Vandenberghe, J. J. Rux, R. Calcedo, J. Sanmiguel, Z. Abbas, and J. M. Wilson. 2003. Adeno-associated viruses undergo substantial evolution in primates during natural infections. Proc Natl Acad Sci USA 100:6081-6.
Gao, G., L. H. Vandenberghe, M. R. Alvira, Y. Lu, R. Calcedo, X. Zhou, and J. M. Wilson. 2004. Clades of Adeno-associated viruses are widely disseminated in hurnan tissues. J Virol 78:6381-8.
Girod, A., C. E. Wobus, Z. Zadori, M. Ried, K. Leike, P. Tijssen, J. A. Kleinschmidt, and M. Hallek. 2002. The VP1 capsid protein of adeno-associated virus type 2 is carrying a phospholipase A2 domain required for virus infectivity.
Gregorevic, P., Blankinship, M. J., Allen, J. M., Crawford, R. W., Meuse, L., Miller, D. G., Russell, D. W. and Chambelin, J. S., “Systemic delivery of genes to striated muscles using adeno-associated viral vectors”, Nature Med. 10, 828-834 (2004).
Grimm, D., Kern, A., Rittner, K., and Kleinschmidt, J. A., Novel tools for production and purification of recombinant adeno-associated virus vectors, Hum Gene Ther. 9, 2745-2760 (1998).
Hauck, B., L. Chen, and W. Xiao. 2003. Generation and characterization of chimeric recombinant AAV vectors. Mol Ther 7:419-25.
Hildinger, M., A. Auricchio, G. Gao, L. Wang, N. Chirmule, and J. M. Wilson. 2001. Hybrid vectors based on adeno-associated virus serotypes 2 and 5 for muscle-directed gene transfer. J Virol 75:6199-203.
Kool, M., P. M. van den Berg, J. Tramper, R. W. Goldbach, and J. M. Vlak. 1993. Location of two putative origins of DNA replication of Autographa californica nuclear polyhedrosis virus. Virology 192:94-101.
Kotin, et al., WO 03/042361, published May 22, 2003.
Kotin, et al., US application 20040197895, 2004.
McCarty, D. M., M. Christensen, and N. Muzyczka. 1991. Sequences required for coordinate induction of adeno-associated virus p 19 and p40 promoters by Rep protein. J Virol 65:2936-45.
O'Reilly, D. R., Miller, L. K., Luckow, V. A. (1994) Baculovirus expression vectors: a laboratory manual.
Potter, M., K. Chesnut, N. Muzyczka, T. Flotte, and S. Zolotukhin. 2002. Streamlined large-scale production of recombinant adeno-associated virus (rAAV) vectors. Methods Enzymol 346:413-30.
Rabinowitz, J. E., D. E. Bowles, S. M. Faust, J. G. Ledford, S. E. Cunningham, and R. J. Samulski. 2004. Cross-dressing the virion: the transcapsidation of adeno-associated virus serotypes functionally defines subgroups. J Virol 78:4421-32.
Rabinowitz, J. E., F. Rolling, C. Li, H. Conrath, W. Xiao, X. Xiao, and R. J. Samulski. 2002. Cross-packaging of a single adeno-associated virus (AAV) type 2 vector genome into multiple AAV serotypes enables transduction with broad specificity. J Virol 76:791-801.
Sanders, D. A., “No false start for novel pseudotyped vectors” Current Opinion in Biotechnology 13: 437-442 (2002).
Urabe, M., C. Ding, and R. M. Kotin. 2002. Insect cells as a factory to produce adeno-associated virus type 2 vectors. Hum Gene Ther 13:1935-43.
Urabe, M., T. Nakakura, K. Ozawa, and R. M. Kotin. 2004. Production of Recombinant Adeno-Associated Virus Type 5 in Insect cells. Mol Ther 9:S160.
Wobus, C. E., B. Hugle-Dorr, A. Girod, G. Petersen, M. Hallek, and J. A. Kleinschmidt. 2000. Monoclonal antibodies against the adeno-associated virus type 2 (AAV-2) capsid: epitope mapping and identification of capsid domains involved in AAV-2-cell interaction and neutralization of AAV-2 infection. J Virol 74:9281-93.
Xiao, X., J. Li, and R. J. Samulski. 1998. Production of high-titer recombinant adeno-associated virus vectors in the absence of helper adenovirus. 1 Virol 72:2224-32.
Zadori, Z., J. Szelei, M. C. Lacoste, Y. Li, S. Gariepy, P. Raymond, M. Allaire, I. R. Nabi, and P. Tijssen. 2001. A viral phospholipase A2 is required for parvovirus infectivity. Dev Cell 1:291-302.
Zolotukhin, S., B. J. Byrne, E. Mason, 1. Zolotukhin, M. Potter, K. Chesnut, C. Summerford, R. J. Samulski, and N. Muzyczka. 1999. Recombinant adenoassociated virus purification using novel methods improves infectious titer and yield. Gene Ther 6:973-85.

What is claimed is:

1. A method for producing recombinant adeno-associated virus (rAAV) in an insect cell, comprising,
A) co-infecting an insect cell with four separate vectors, each vector comprising:
i) a nucleotide sequence encoding a baculovirus Rep78 or Rep68 operably linked to an expression control sequence for expression in the insect cell;
ii) a nucleotide sequence encoding a baculovirus Rep52 or Rep40 operably linked to an expression control sequence for expression in the insect cell;
iii) a nucleotide sequence encoding a VP1 AAV capsid protein operably linked to a promoter; and
iv) a recombinant AAV expression vector comprising a selected transgene positioned between two AAV inverted terminal repeat sequences; and
B) maintaining the insect cell under incubation conditions favorable for rAAV production to produce rAAV.

2. The method of claim 1 wherein the insect virus is a baculovirus.

3. The method of claim 1 wherein the expression control sequence is a strong viral promoter.

4. The method of claim 1 wherein the strong viral promoters are selected from the group consisting of pohl, p10, CaMV, CMV5, EBVqp, 35S, AdMLP, BM5, HLH, CDK9, HCMVie, IE-1, and HIV-1LTR.

5. The method of claim 1 wherein the expression control sequence operably linked to the nucleic acid sequence encoding Rep52 is pohl.

6. The method of claim 1 wherein the expression control sequence operably linked to the nucleic acid sequence encoding Rep 78 is a Δ1E-1promoter.

7. The method of claim 1 wherein the promoter operably linked to the nucleic acid encoding VP1 AAV capsid protein is a pohl promoter.

8. The method of claim 1 wherein the expression vector comprises a p10 promoter.

9. The method of claim 5 wherein the insect cell is selected from the group consisting of Anticarsia gemmatalis MNPV, Agrotis ipsilon nucleopolyhedrovirus, Autographa california MNPV, Bombyx mori NPV, Buzura suppressaria nucleopolyhedrovirus, Choristoneura fumiferana MNPV, Choristoneura fumiferana DEF nucleopolyhedrovirus, Choristoneura rosaceana nucleopolyhedrovirus, Culex nigripalpus nuclepoolyhedrovirus, Epiphyas postvittana nucleopolyhedrovirus, Helicoverpa armisgera nucleopolyhedrovirus, Helicoverpa zea single nucleopolyhedrovirus, Lymantria dispar MNPV, Mamestra brassicae MNPV, Mamestra configurata nucleopolyhedrovirus, Neodiprion lecontii nucleopolyhedrovirus, Neodiprion sertifer NPV, Orgyia pseudotsugata MNPV, Spodoptera exigua MNPV, Spodoptera frugiperda MNPV, Spodoptera littoralis nucleopolyhedrovirus, Thysanoplusia orichalcea nucleopolyhedrovirus, Trichoplusia ni single nucleopolyhedrovirus, Wiseana signata nucleopolyhedrovirus.

10. The method of claim 1 wherein the insect cell is Sf9 or Sf21.

11. The method of claim 10 wherein the insect cell is Sf9.

12. The method of claim 1 wherein the encoded AAV capsid protein is selected from the group consisting of AAV4, AAV5, AAV6, AAV7 and AAV8.

13. The method of claim 12 wherein the encoded capsid proteins are AAV2/5.

14. A method for sustained high titer rAAV production, comprising:
(a) co-infecting an insect cell with the four vectors of claim 1;
(b) incubating the cell in suitable media for a period of time sufficient to produce at least 1×10⁴ AAV particles per cell;
(c) infecting a second insect cell with media from step b); and
(d) repeating steps (a)-(c) wherein sustained high titer rAAV production is obtained through at least five consecutive passages.

15. A recombinant insect virus vector comprising two nucleic acid sequence open reading frames (ORFs) encoding a Rep52 or a Rep 48 and a BacVP positioned tail-to-tail and operably linked to an expression control sequence for expression in an insect cell, wherein said vector sustains expression of Rep52 or Rep 48 and BacVP through multiple passages of insect cell infections.

16. A recombinant insect virus vector comprising two nucleic acid sequence open reading frames (ORFs) encoding a Rep78 or a Rep 68 and a BacVP positioned tail-to-tail and operably linked to an expression control sequence for expression in an insect cell wherein said vector sustains expression of Rep78 or Rep68 and BacVP through multiple passages of insect cell infections with said vector.

17. The recombinant insect virus vector of claim 16 wherein the BacVP comprises a chimeric AAV V1 protein partially substituted with an AAV phospholipid domain.

18. The recombinant insect virus vector of claim 17 wherein the AAV phospholipid domain is AAV phospholipase A2.

19. An insect cell comprising the recombinant vector of claim 15 and claim 16.

20. An insect cell of comprising the recombinant vector of claim 15 or claim 16

21. An insect cell comprising the recombinant vector of claim 18.

22. The insect cell of claim 14 identified as an Sf9 cell.

23. A method for preparing pseudotyped rAAV, comprising the method of claim 1 wherein the nucleic acid sequence encoding the AAV V1 capsid protein comprises a parvoviral phospholipid domain.

24. The method of claim 24 wherein the parvoviral phospholipid domain is VP1 phospholipase A2 (pvPLA2).

25. The method of claim 23 wherein the pseudotyped rAAV capsid comprises the amino acid sequence of SEQ ID NO: 1.

26. The method of claim 23 wherein the pseudotyped rAAV capsid comprises the amino acid sequence of SEQ ID NO: 2.

27. The method of claim 23 wherein pseudotyped rAAV capsid comprises the amino acid sequence of SEQ ID NO: 3.

28. Recombinant pseudotyped adeno-associated virus prepared by the method of claim 13 or claim 24.

29. The recombinant pseudotyped adeno-associated virus of claim 28, which efficiently transduces to liver cells in vivo.


SEQUENCE LISTING (3 sequences)

SEQ ID NO: 1 - protein, 137 aa, adeno-associated virus
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVK

SEQ ID NO: 2 - protein, 137 aa, adeno-associated virus
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAK

SEQ ID NO: 3 - protein, 137 aa, Artificial Sequence
  Description of Artificial Sequence Chimeric AAV2/8 peptide
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEGAK